(19) United States
(12) Patent Application Publication
(10) Pub. No.: US 2022/0280101 A1
(43) Pub. Date: Sep. 8, 2022

(54) WIRELESS PRESSURE-SENSOR INTEGRATED SMART BANDAGE

(71) Applicant: The Curators of the University of Missouri, Columbia, MO (US)

(72) Inventors: Xueju Wang, Columbia, MO (US); Carolyn Crumley, Lee's Summit, MO (US)

(73) Assignee: The Curators of the University of Missouri, Columbia, MO (US)

(21) Appl. No.: 17/687,163

(22) Filed: Mar. 4, 2022

Related U.S. Application Data
(60) Provisional application No. 63/156,862, filed on Mar. 4, 2021.

(51) Int. Cl. A61B 5/00; A61F 13/06; A61L 15/42; A61L 15/58

(52) U.S. Cl. CPC A61B 5/445; A61B 5/746; A61B 5/742; A61B 5/0004; A61B 5/6829; A61F 13/069; A61L 15/425; A61L 15/58; A61B 2562/0247

(57) ABSTRACT

The present disclosure generally relates to smart bandages comprising a dressing and pressure sensor. The pressure sensor measures at least one pressure reading at a wound area while the dressing bandages the wound area. The pressure sensor includes a force sensitive film configured to generate a pressure signal based on pressure applied thereto and is operably coupled to the dressing such that pressure applied to the wound area is coupled to the force sensitive film via the dressing. A near field communication device of the pressure sensor is operably coupled to the force sensitive film for reporting the pressure signal. The smart bandage may include a user interface that is configured to display reported signals to a user for comparing the pressure at the wound area with a threshold pressure. The disclosure further provides a method of treating the wound area by applying the smart bandage to the wound area.

### CROSS-REFERENCE TO RELATED APPLICATIONS

This application claims the benefit of U.S. Provisional Patent Application No. 63/156,862, filed Mar. 4, 2021, the entire contents of which are incorporated herein by reference.

### FIELD

The present disclosure generally relates to smart bandages comprising pressure sensors and methods that provide for enhanced wound monitoring and healing.

### BACKGROUND

Pressure is a contributing factor to the development of many wounds, including diabetic foot ulcers and pressure injuries. Ongoing, unrelieved pressure results in delayed wound healing and recurrence. Offloading of pressure is typically achieved by instructing the patient to avoid pressure to the area, and the use of adaptive devices to reduce or eliminate pressure. Adherence to offloading measures has been demonstrated to be very low. Currently available pressure mapping devices are designed for isolated usage within clinical settings, to measure pressure at a single point in time, and must be interpreted by a trained clinician. Products are needed that provide continuous real-time monitoring of pressure, with feedback to the patient to encourage adherence to offloading measures.

### BRIEF SUMMARY

In one aspect, the present disclosure provides a smart bandage that includes a dressing and a pressure sensor. The pressure sensor is coupled to the dressing to be positioned on a wound area while the dressing bandages the wound area. The pressure sensor is configured to measure at least one pressure reading at the wound area. The pressure sensor includes a force sensitive film, a near field communication device, and a battery. The force sensitive firm is configured to generate a pressure signal based on pressure applied thereto and is operably coupled to the dressing such that pressure applied to the wound area is coupled to the force sensitive film via the dressing. The near field communication device is operably coupled to the force sensitive film for reporting the pressure signal. The battery is configured to provide power to the pressure sensor.

In another aspect, the present disclosure provides a method of treating a wound area of a patient by applying the smart bandage to the wound area of the patient, measuring one or more pressure readings at the wound area using the pressure sensor, and transmitting the one or more pressure readings to a user interface using the near field communication device.

In yet another aspect, the present disclosure provides a smart bandage further including the user interface that is configured to display reported signals to a user for comparing the pressure at the wound area with a threshold pressure. The threshold pressure is indicative of too much pressure loaded on the wound area. The smart bandage further includes a circuit board coupling a connecting housing, general circuit components, data processing and transmission components. The connecting housing electrically couples the pressure sensor to the circuit board. The general circuit components comprise an operational amplifier to amplify and transmit analog data from the pressure sensor to the data processing and transmission components. The data processing and transmission components read and extract data from the pressure signal and transmit the data to the user interface.

### BRIEF DESCRIPTION OF THE DRAWINGS

FIG. 1 is a top view of a smart bandage according to one aspect of the disclosure;

FIG. 2 a bottom view of a user's foot with smart bandages coupled to the foot;

FIG. 3 an illustration of a user's foot with a smart bandage on the bottom on the foot with a portion extending on the top of the foot;

FIG. 4 is a schematic of a circuit diagram of the smart bandage;

FIG. 5 is a top view of a printed circuit board of the smart bandage;

FIG. 6 is a bottom view of the printed circuit board of the smart bandage;

FIG. 7 is an illustration of the smart bandage and a user interface;

FIG. 8 is an exploded view of a smart bandage according to one aspect of the disclosure;

FIG. 9 is a top view of an embodiment of the smart bandage on a user's foot;

FIG. 10 is exploded view of a pressure sensor according to one aspect of the disclosure;

FIG. 11 is a perspective view of a force sensitive film according to one aspect of the disclosure;

FIG. 12 is an isolated view of a force sensitive film according to one aspect of the disclosure;

FIG. 13 is an example reading and report workflow for a smart bandage according to aspects of the disclosure;

FIG. 14 is an illustration of loading the smart bandage of FIG. 3;

FIG. 15A is an illustration of the user interface indicating sufficient offloading;

FIG. 15B is an illustration of the user interface indicating inadequate offloading;

FIG. 16A is an illustration of the user interface displaying data from a session duration;

FIG. 16B is an illustration of the user interface exporting data from the session duration;

FIG. 17 is an illustration of locations for use of the smart bandage according to aspects of the disclosure;

FIG. 18 shows the results of pressure studies of embodiments of the disclosure;

FIG. 19A and FIG. 19B is a graphical representation of a study for fractional of resistance throughout increasing applied pressures on the smart bandage;

FIG. 19C and FIG. 19D is a graphical representation of a study for realtime converted pressure over time compared to the real pressure applied to the device;

FIG. 20A is a schematic illustration of applying the smart bandage on a participant under a medical grade boot;

FIG. 20B is a graphical representation of data collected from smart bandage of FIG. 20A with the participant in a standing position;

FIG. 20C is a graphical representation of data collected from smart bandage of FIG. 20A with the participant walking;

FIG. 20D is a graphical representation of data collected from a heel of a participant with the participant in a standing position.

FIG. 20E is a graphical representation of data collected from the heel with the participant walking.

FIG. 21A is a graphical representation analog-to-digital averages measures precalibration and after calibration for three different smart bandages;

FIG. 21B is a graphical representation of pressure measurements of three different smart bandages of FIG. 21A after calibration;

FIG. 22 is a graphical representation of precalibrated pressure measurements of a pressure sensor of a smart bandage inside and outside the smart bandage and a calibrated pressure measurement inside the smart bandage;

FIG. 23A is a graphical representation of a response time of the pressure sensor of the smart bandage with extracted views of the onloading and offloading responses;

FIG. 23B is a graphical representation of pressure of the pressure sensor of the smart bandage during a period of cyclic loads (n=100) with an extracted view of two cycles;

FIG. 23C is a graphical representation of fractional changes in resistance of the pressure sensor of the smart bandage over an extended period during complete submersion in water (n=3);

FIG. 23D is a graphical representation of fractional changes in resistance of the pressure sensor of the smart bandage over a period of increasing temperatures in an oven (n=3).

Corresponding reference characters indicate corresponding parts throughout the drawings.

### DETAILED DESCRIPTION

In general, the systems described herein are smart bandages designed for use on a patient for covering a wound and monitoring pressure on the wound. The smart bandage monitors pressure in real-time, improving patient adherence to offloading recommendations. This provides accelerated healing rates, prevents complications and amputations, and saves medical costs. The smart bandages also enable clinicians to track the pressure record of patients and quickly pinpoint the higher-risk patients with inadequate offloading, which will assist in making medical decisions for treatment.

Some aspects are directed to methods of using the smart bandages for treating a wound area of a patient. In some aspects, the wound areas are diabetic foot ulcers.

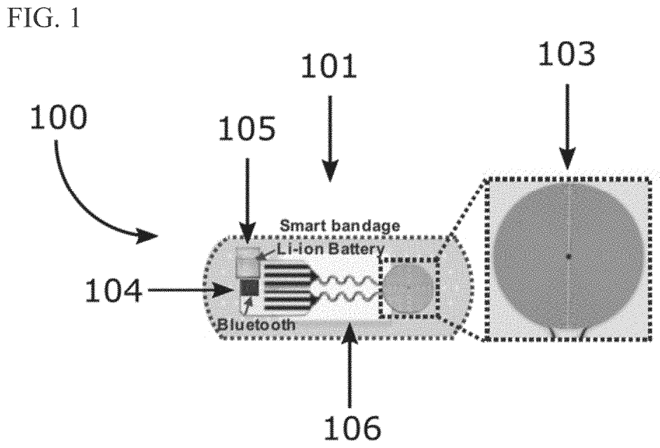

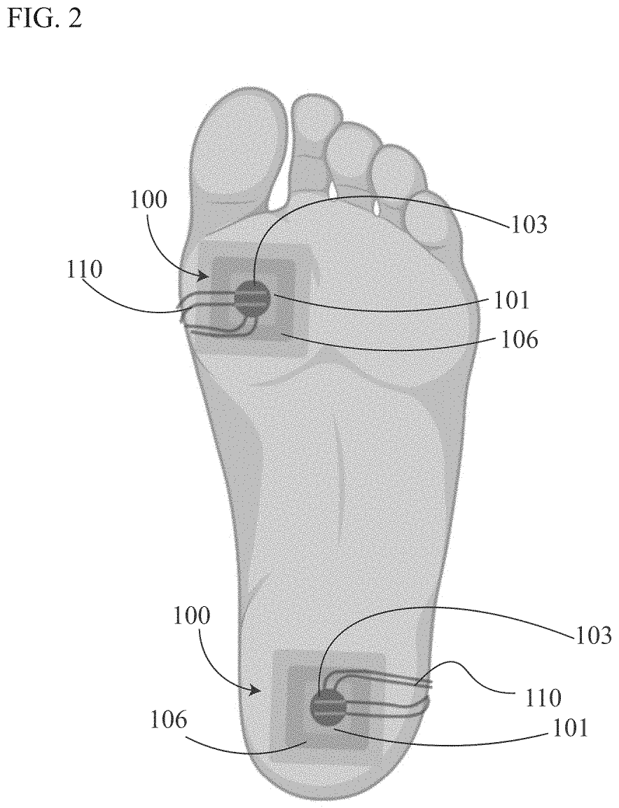

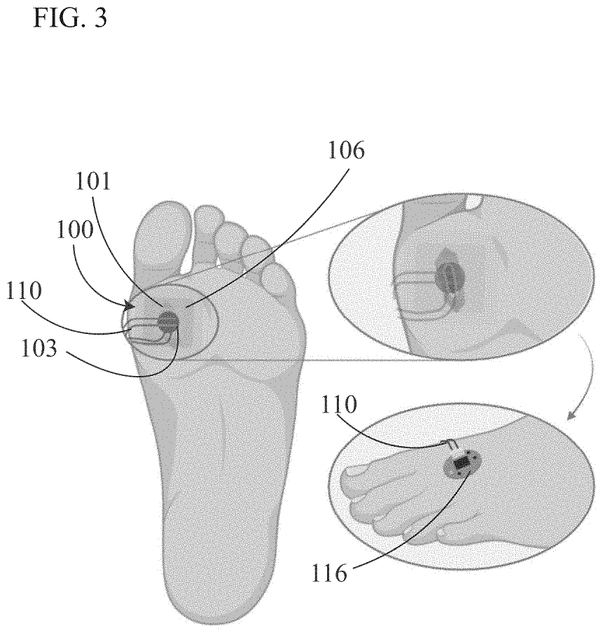

Turning to the figures, FIG. 1 shows a smart bandage, generally indicated at 100, according to one aspect of the disclosure. The smart bandage 100 includes a pressure sensor 101 for monitoring a pressure on a wound on a user, as shown in FIGS. 2-3. The pressure sensor 101 is small and flexible such that the smart bandage 100 is comfortable for the patient, even during movement. In an embodiment, the pressure sensor 101 includes a force sensitive film 103, a near field communication device 104 (FIG. 1), and a battery 105 (FIG. 1). The force sensitive film 103 generates a signal based on pressure on the force sensitive film 103, and the pressure sensor 101 is configured such that the signal from the force sensitive film 103 is communicated to the near field communication device 104. The near field communication device 104 reports the pressure readings of the pressure sensor. For example, in some embodiments the near field communication device 104 is a Bluetooth device. In some embodiments, the pressure sensor 101 detects pressure by a change in a geometry of the force sensitive film 103 which changes the resistance of the force sensitive film 103. In some embodiments, the pressure sensor 101 is a conformal resistive pressure sensor. By calibrating the pressure sensor 101 to a consistent reference at a zero load, alternative pressure sensor types may be used with consistency in percentage changes once pressure is applied. The battery 105 is configured to provide power to the pressure sensor 101. In some embodiments, the battery 105 is a lithium-ion battery.

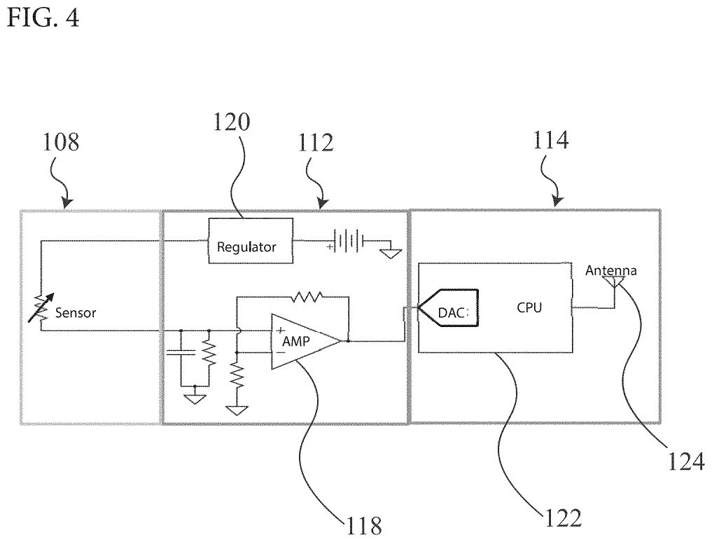

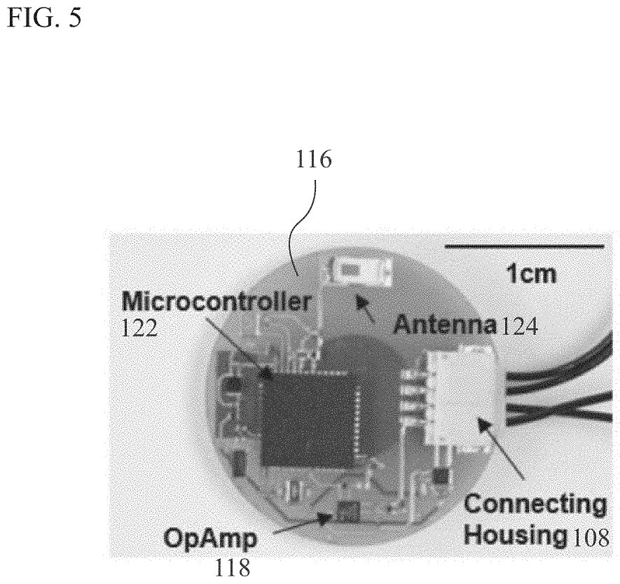

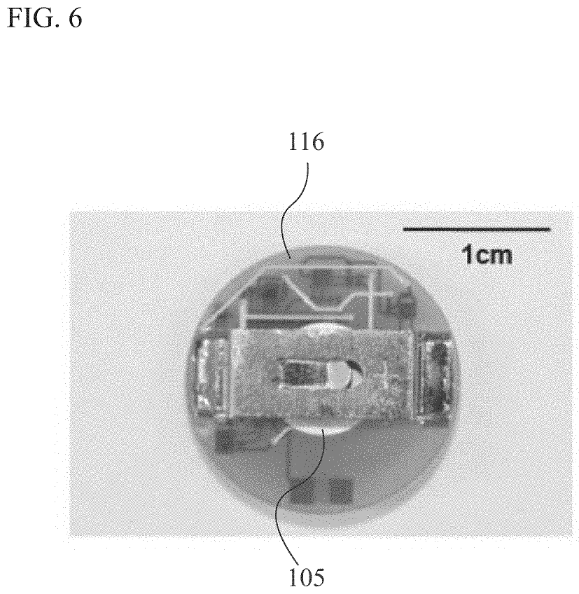

In an embodiment, as shown in FIGS. 4-6, the smart bandage 100 includes a connecting housing 108, general circuit components 112, and data processing and transmission components 114 on a printed circuit board 116. The connecting housing 108 connects the pressure sensor 101 to the printed circuit board 116 such that the pressure sensor may be distanced from the printed circuit board 116 by wires (e.g., conductive threads 110). Distancing the pressure sensor 101 from the printed circuit board 116 allows the pressure sensor to be on the wound area (e.g., diabetic foot ulcer) while rigid components on the printed circuit board are located on a nonimpact site to avoid further injury to the wound area by contact with the rigid components, as shown in FIG. 3. The general circuit components 112 include an operational amplifier (OpAMP) 118 and regulator 120 to amplify and transmit analog data from the pressure sensor 101 to the data processing and transmission components 114. The data processing and transmission components 114 include a microcontroller 122 and antenna 124. The microcontroller 122 controls processing and transmitting of the analog data by processing the data by a digital to analog converter and a central processing unit to read and extract data from the pressure sensor 101, which will be sent to the user on a user interface 126. In the embodiment shown, the printed circuit board 116 of the smart bandage 100 includes a top face and a bottom face with the top face coupling the connecting housing 108 general circuit components 112, and data processing and transmission components 114 to the printed circuit board and the bottom face coupling the battery 105. It is to be understood that battery 105 may be located spaced apart from smart bandage 100 to enhance patient comfort.

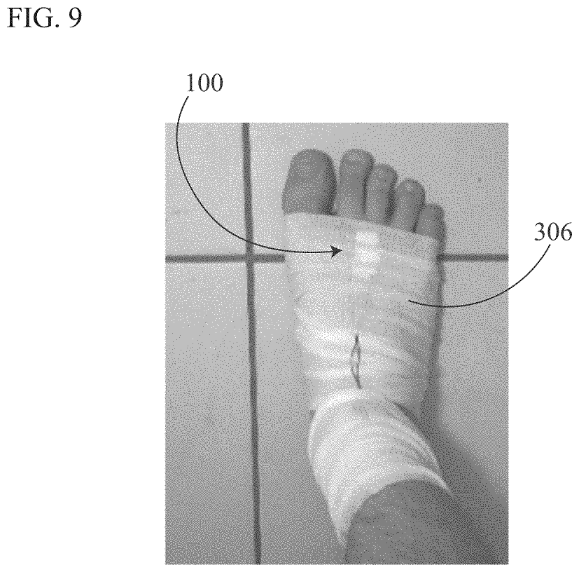

As shown in FIGS. 1-3 and 8, the smart bandage 100 also includes a dressing 106, 206 for dressing the wound. The dressing 106, 206 may be used to secure the pressure sensor 101, 201 while aiding in healing of the user by dressing the wound. In some embodiments, the pressure sensor 101 is within the dressing 106, 206 such that the dressing has direct contact with wound while the pressure sensor has contact through the dressing. In some embodiments, as shown in FIG. 9, the dressing 306 (e.g., gauze, athletic tape) may be wrapped over the smart bandage 100. In an embodiment, a walking boot or shoe may be worn over the smart bandage 100.

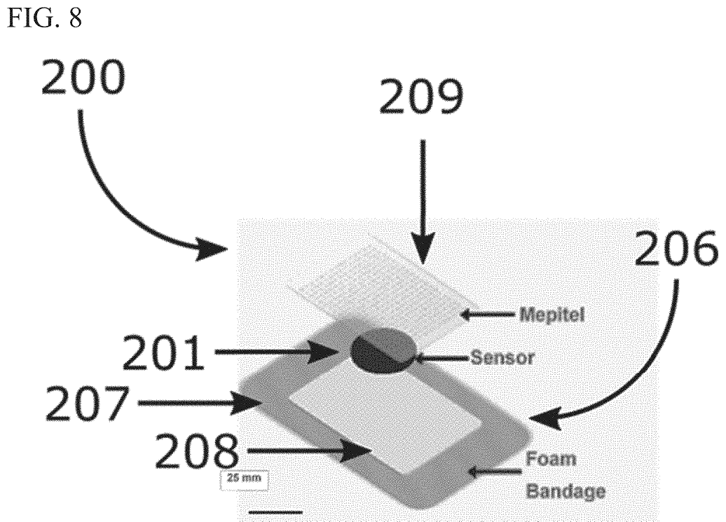

In an embodiment, as shown in FIG. 8, the dressing 206 of a smart bandage 200 includes an adhesive layer 207 and a foam layer 208. For example, in some embodiments the dressing 206 is or includes a foam bandage. The smart bandage 200 includes a porous adhesion layer 209. In some embodiments, the pressure sensor 201 is between the porous adhesion layer 209 and the dressing 206.

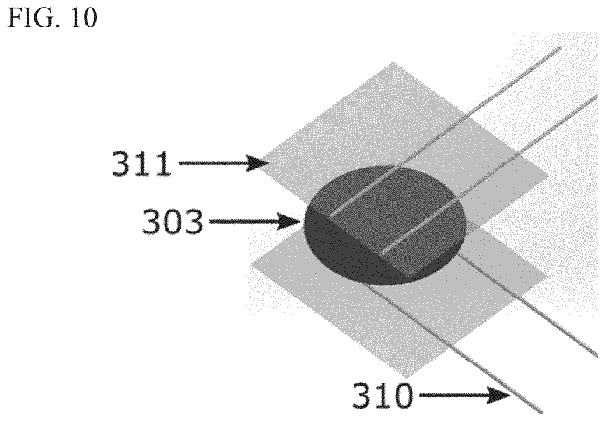

Smart bandages according to various embodiments further include one or more covering layers covering a surface of the force sensitive film. In various embodiments, the pressure sensor 101 further includes conductive threads 110 for communication between the pressure sensor and the communication device, as shown in FIGS. 2-3 and 7. As shown in FIG. 10, a first of one or more conductive threads 310 is between a top side of a force sensitive film 303 and a covering layer 311, and a second of the one or more conductive threads 310 is below a bottom side of the force sensitive film 303 and a second covering layer 311. In some embodiments, the covering layers 311 have a thickness of about 5 μm to about 250 μm. In some embodiments, the covering layers 311 include a polymer. In some embodiments, the covering layers comprise polyimide, SU-8 or polydimethylsiloxane. In some embodiments, the force sensitive film 303 is between two or more covering layers 311. In some embodiments, the one or more covering layers laminate the force sensitive film. In an embodiment, a silicon adhesive (e.g., KWIK-SIL) may be used to further seal the pressure sensor 101 in the case of excessive laminates. In some embodiments, the pressure sensor 101 has thickness of from about 0.1 to about 1 mm, from about 0.2 to about 0.8 mm, or from about 0.3 to about 0.5 mm.

The flexibility and comfort of the pressure sensor 101 of the smart bandage 100 in part enabled by the thin, flexible, force sensitive film. The flexibility of the pressure sensor 101 by the force sensitive film 103 mitigates mechanical mismatch between the sensor and skin surface. In some embodiments, the force sensitive film has a thickness of about 0.3 mm or less, about 0.2 mm or less, or about 0.1 mm or less. In some embodiments, the force sensitive film has a diameter about 10 mm to about 40 mm. In some embodiments, the force sensitive film has a diameter of about 20 mm to about 30 mm. In some embodiments, the force sensitive film has a volume resistivity of about 500 ohm-cm or less. In some embodiments, the force sensitive film has a surface resistivity of about 31,000 ohms/sq·cm or less.

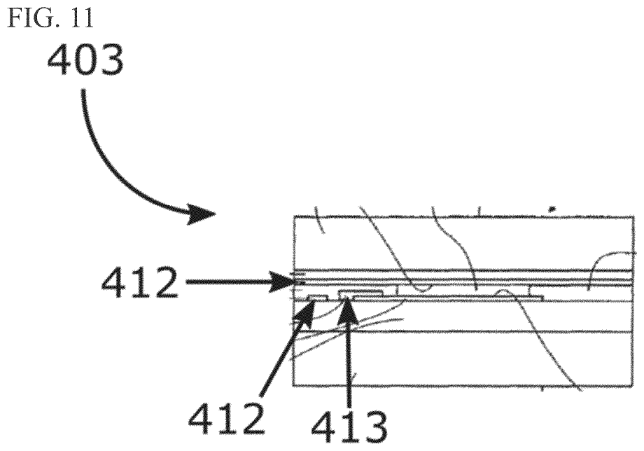

FIG. 11 shows a side layer view of a force sensitive film 403 in accordance with various embodiments of the disclosure. Under pressure, electrodes 412 are pressed towards each other. The area of contact between the electrodes 412 will increase with increasing applied pressure, thereby providing a shunt path past the conduction path provided by the resistive element 413. Hence, the impedance of the pressure sensor will decrease stepwise as a function of the applied pressure.

In some embodiments, force sensitive film comprises a planar metal film. In some embodiments, the electrodes 412 are thin metal films. In various embodiments, the planar metal film has a thickness of about 400 nm or less, about 300 nm or less, or about 200 nm or less. In various embodiments, the planar metal film comprises gold and/or platinum.

In some embodiments, the force sensitive film further comprises a polymer layer. In various embodiment, the resistive element 413 is a polymer layer. In various embodiments, the force sensitive film comprises a plurality of planar metal films and a plurality of polymer layers.

FIG. 12 shows a zoomed in view of a force sensitive film 503 in accordance with preferred embodiments. The force sensitive film 503 comprises an electrically conductive polymer. Under pressure, the relative positions of the particles 514 present in the force sensitive film 503 will change, thereby changing the impedance of the pressure sensor. In some embodiments, the electrically conductive polymer comprises electrically conductive carbon or carbon black. In some embodiments, the force sensitive film 503 comprises a carbon-impregnated polyolefin.

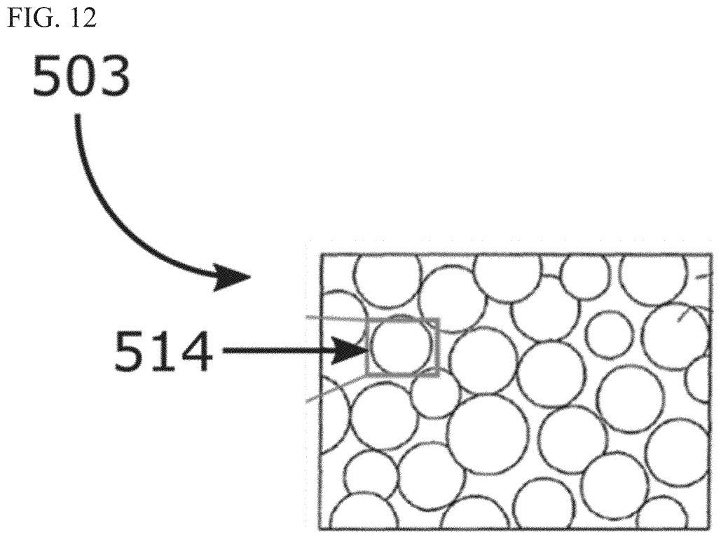

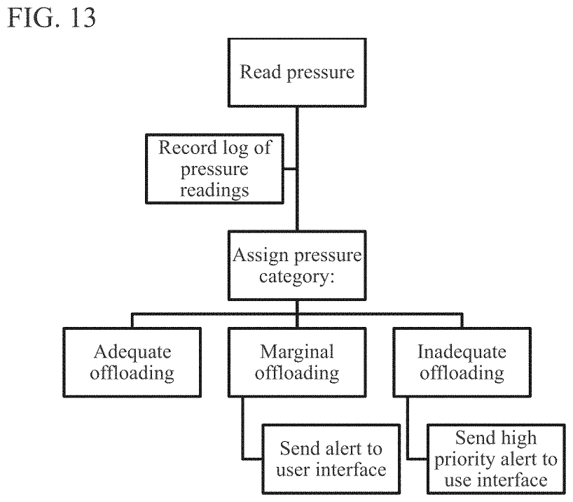

In various embodiments, the smart bandage 100 is configured to relay to a user interface using the near field communication device 104. In some embodiments, the smart bandage is configured to measure a pressure reading and assign the pressure reading to a category selected from the group consisting off 1) adequate offloading, 2) marginal offloading, and 3) inadequate offloading. For example, in some embodiments the smart bandage is configured to send an alert to a user interface if a pressure reading is assigned to inadequate offloading. In various embodiments the smart bandage is configured to continuously measure pressure readings, and in some embodiments the user interface maintains a log of the pressure readings. An example reading and report workflow is shown in FIG. 13.

Smart bandages in accordance with various embodiments are used in methods for treating a wound area of a patient. These methods include applying the smart bandage to the wound area of the patient; measuring one or more pressure readings at the wound area using the pressure sensor; and transmitting the one or more pressure readings to a user interface using the near field communication device. Some embodiments of the method further includes cutting the smart bandage to a shape corresponding to the wound area or based on the shape of the wound area. In various embodiments the methods further include reporting an offloading suggestion based on the one or more pressure readings to an end user using the user interface.

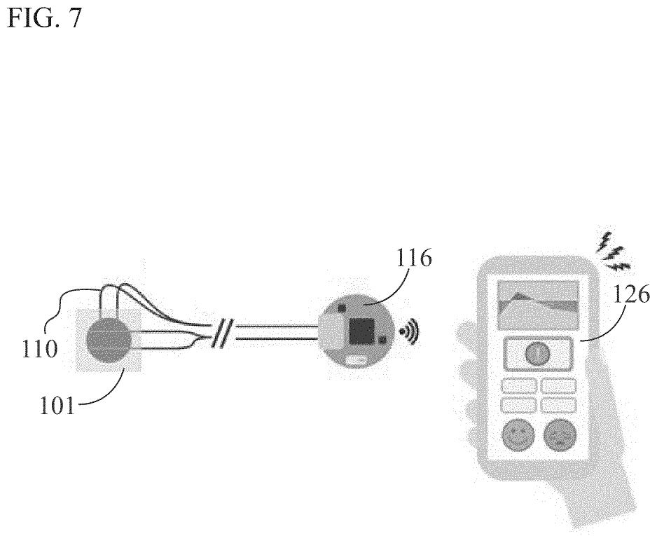

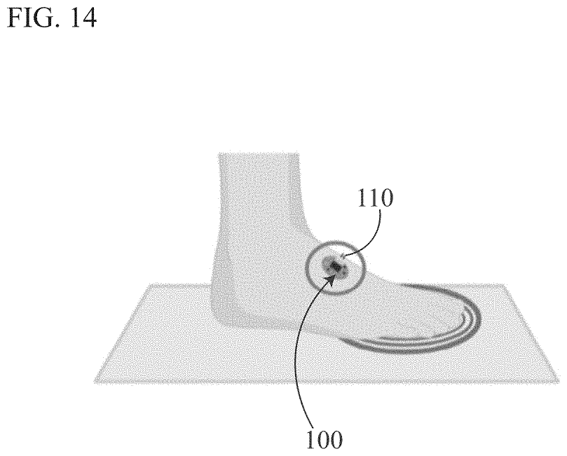

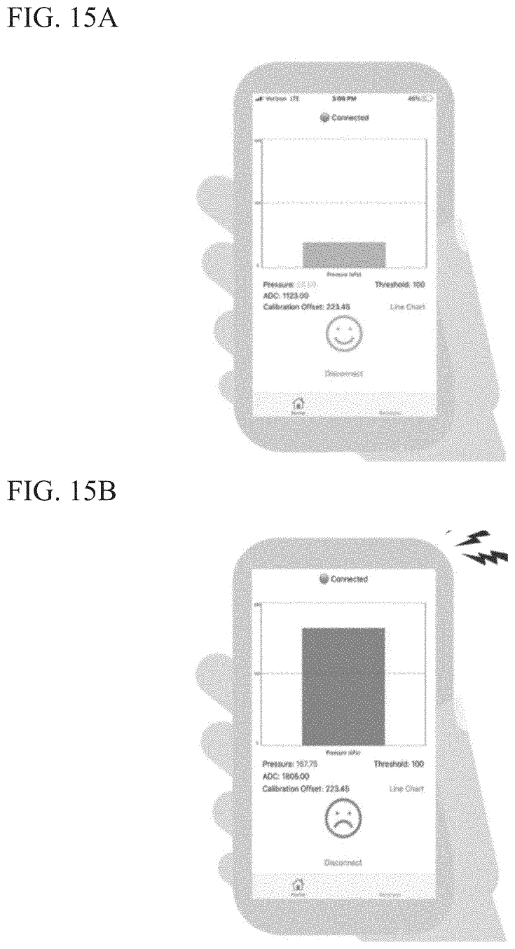

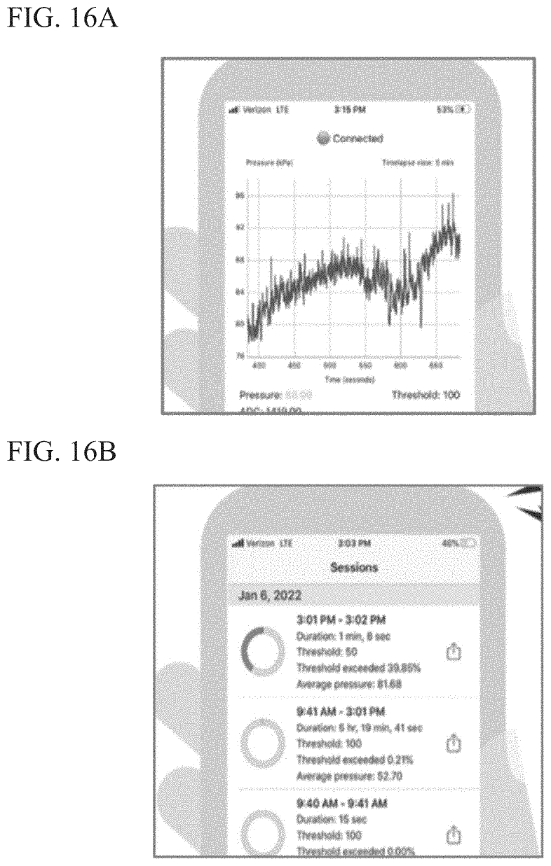

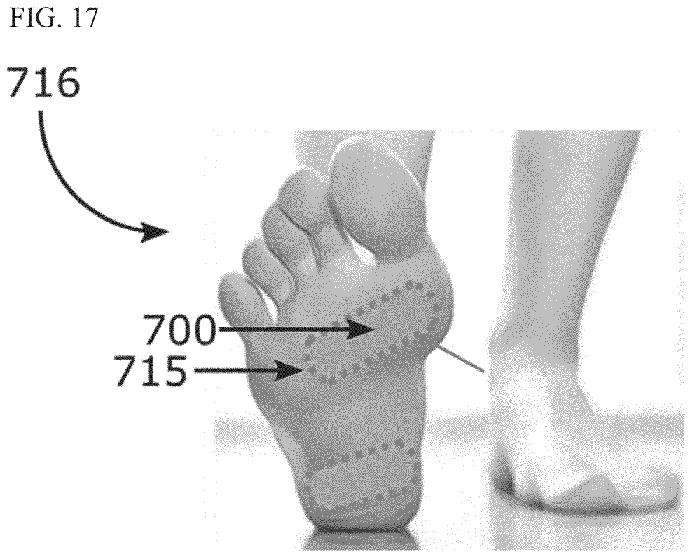

As shown in FIG. 7, the user interface may be an IoS Application Interface (e.g., user interface 126) to display pressure from the pressure sensor. The user interface 126 may report a real-time pressure exerted on the pressure sensor 101, as shown in FIG. 14, in comparison with a threshold pressure (e.g., a pressure too large on the wound area). Reporting may be through one or both of a numerical value or plotted through a comparison with the threshold pressure, as shown in FIGS. 15A-15B. In an embodiment, when the pressure is plotted in comparison with the threshold pressure, the threshold pressure is displayed by a dashed line along a graph. If the pressure sensor 101 is experiencing a pressure greater than the threshold pressure, offloading needs to occur. Color indicated may be utilized on the user interface 126 by a green display for sufficient offloading and a red display for inadequate offloading. Alternative indications for offloading may be used without departing from the present disclosure. As shown in FIG. 16A, the user interface may report a real-time and an amount of past pressures exerted on the pressure sensor 101 in comparison with a threshold pressure to visualize how pressure on the wound area is changing over time. For a session duration, a highest pressure percentage in reference to the threshold pressure and an average pressure may be calculated and displayed on the user interface 126. The past pressures, highest pressure percentage, and average pressure for the session duration may be exported as session data to a .txt file, as shown in FIG. 16B. As shown in 16B, percentage of time spent with sufficient offloading may be shown through an indication (e.g., the color indication) on the user interface 126.
FIG. 17 shows example application areas 715 on the foot of a patient 716 for smart bandages 700 for treating a wound area of the patient. In preferred embodiments, the patient is a diabetic patient, and the wound area is a diabetic ulcer. In certain embodiments, the wound area is a diabetic foot ulcer. Commonly, diabetic foot ulcers are located on medial planar of the foot and heel. As shown in FIGS. 2-3, the smart bandage 100 is on the mesial planar of the foot and the heel. Placement of the smart bandage 100 may very without departing from the present disclosure.

### Example

The following non-limiting examples are provided to further illustrate the present disclosure.

A soft, flexible pressure sensor 301 was assembled with a tri-layer configuration (FIG. 10). A force sensitive film 303 (FSF, 100 μm thick, Adafruit, New York, N.Y.) was used as the active sensing layer and was patterned using a CO₂ laser (VLS 3.50, Universal Laser System, Norman, Okla.) into geometries that fit the size of diabetic foot ulcers or pressure wounds, about 20 to 30 mm in diameter. In addition, the laser was also used to make markings where conductive threads 310 (used for connection to external equipment for data collection) were integrated onto the FSF to form the pressure sensor. To keep the conductive threads in place, a double-sided tape (236 μm thick, 3M, St. Paul, Minn.) was placed on top of it, followed by encapsulation with a thin polyimide film (125 μm thick) as barriers to fluids. The pressure sensor 301 was then integrated onto a foam bandage (Mepilex Border, Molnlycke, Norcross, Ga.) used for diabetic foot ulcers and covered with a porous adhesion layer 209 (0.5 mm thick, Mepetil, Molnlycke, Norcross, Ga.) as shown generally in FIG. 8.

Evaluation of the Sensor Performance: The performance of the smart bandage was evaluated via testing the smart bandage on 8 healthy individuals. The sensor 301 was applied to the lateral side of their foot (MT1 location), where the foot ulcer usually happens, as shown in FIG. 17. The participants were instructed to stand and do a short walk back and forth in the laboratory. The resistance values of the pressure sensor 301 were recorded using an LCR meter (IM 3533, Hioki, Plano, Tex.) as a function of time as shown in FIG. 18. The participants then wore the smart bandage on their feet for around 6 hours while they performed their regular daily routine. After 6 hours, each pressure sensor 301 and its performance were further evaluated, and was found to have the same performance as when tested initially.

An evaluation form was filled by each individual volunteer after the test was completed. Based on the feedback from the individual, 7 out of 8 individuals found the smart bandage to be very comfortable and to not affect their regular activities.

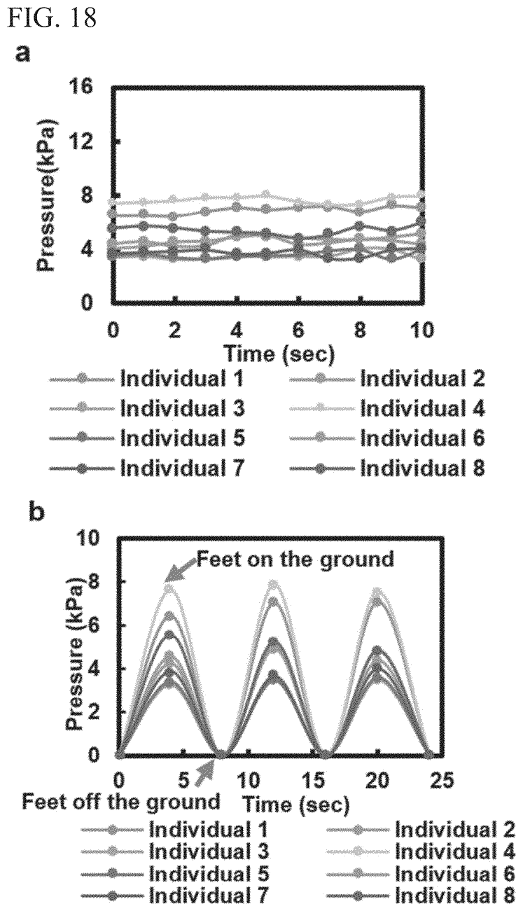

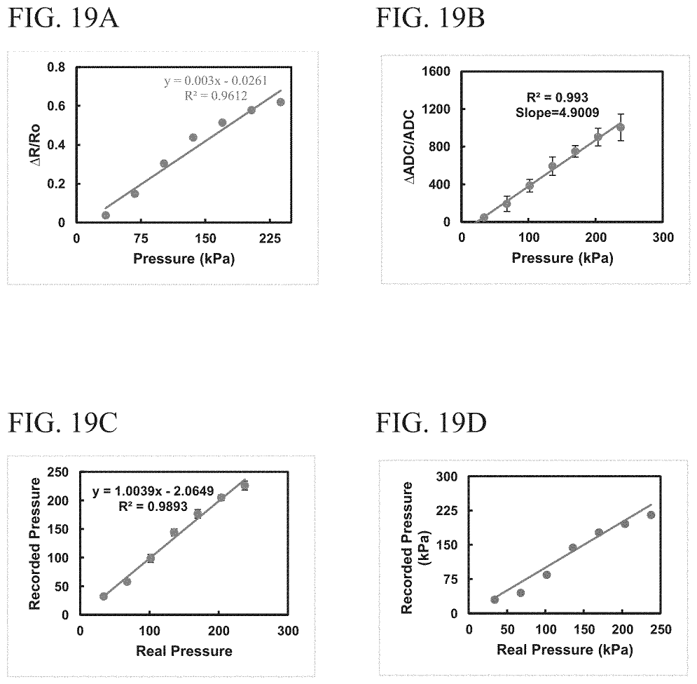

As shown in FIGS. 19A-19B, plots are provided of fractional of resistance throughout increasing applied pressures. As shown in FIGS. 19C-19D, realtime converted pressures over time compared to the real pressures applied to the smart bandage 100.

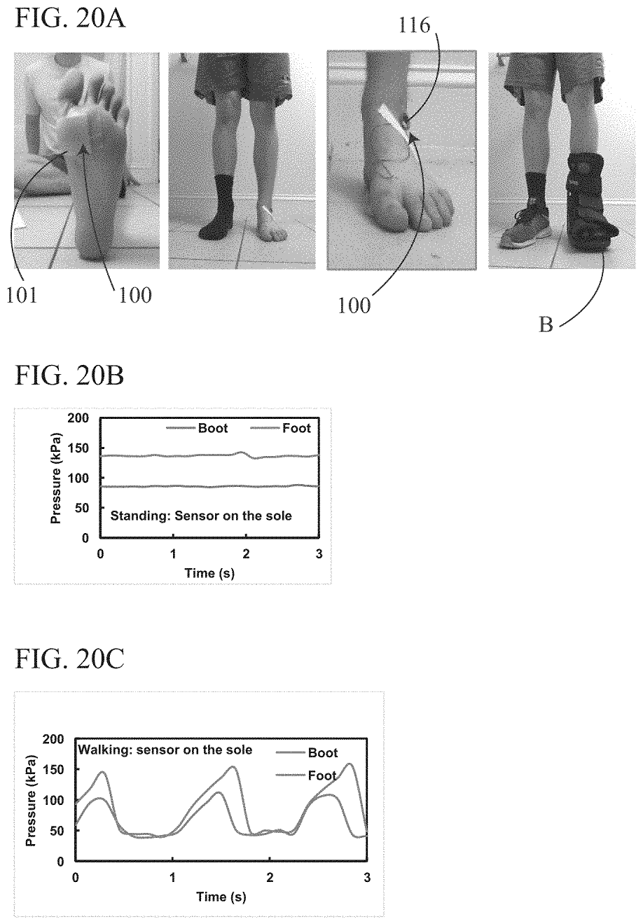

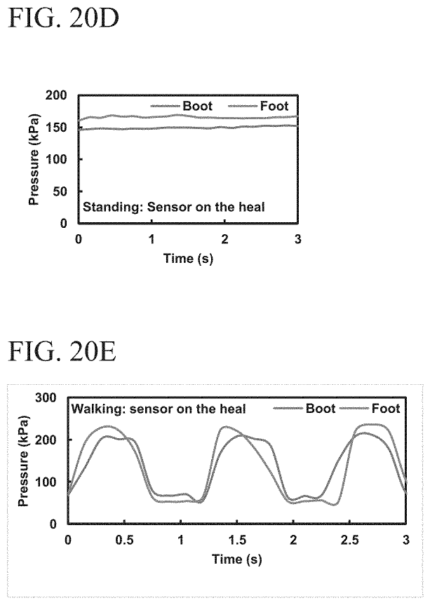

As shown in FIGS. 20A-20E, a study was performed on the smart bandage 100 to determine functionality by comparing pressures exerted on a targeted location (e.g., the wound area being a medial planar area of the participant's foot) through a tennis shoe or a medical grade boot. FIG. 20A shows a study participant wearing the smart bandage 100 with the sensor 101 on the wound area and the printed circuit board 116 away from the wound area (e.g., dorsal area of the participant's ankle) with the medical offloading boot B. FIG. 20B displays data collected from the medial planar with the participant in a standing position. FIG. 20C displays data collected from the medial planar with the participant walking. FIG. 20D displays data collected from a wound area (e.g., the wound area being a heel area of the participant's foot) with the participant in a standing position. FIG. 20E displays data collected from the heel with the participant walking.

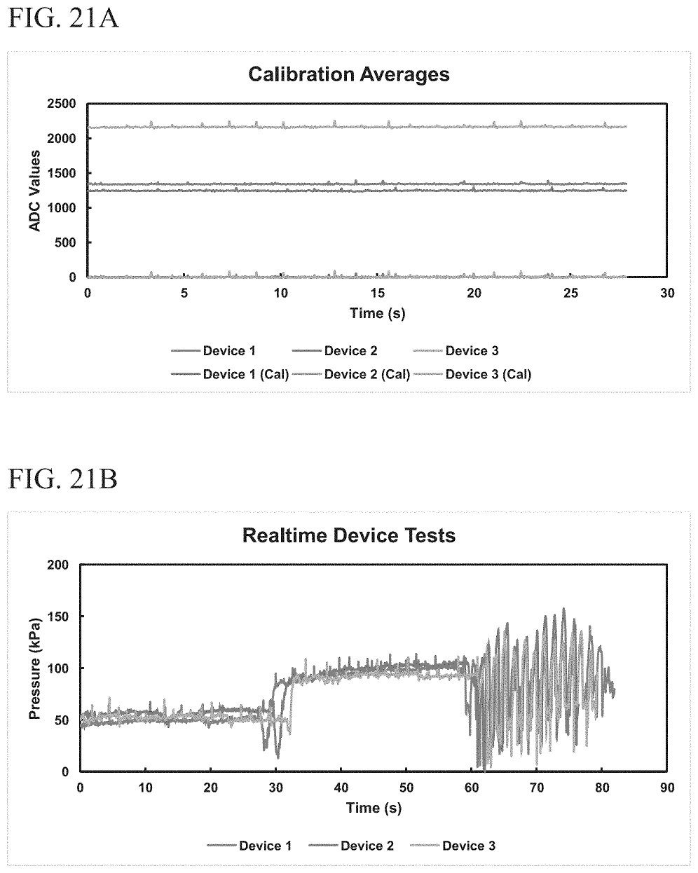

As shown in FIG. 21A, a study was performed on the smart bandage 100 performed with three different sensors (e.g., device 1, device, 2, device 3). Each sensor has a different internal resistance, which means an output voltage at a zero load will be different between each sensor. FIG. 21 displays precalibration and calibration (cal) averages for different sensors reporting various analog-to-digital converter (DAC) values at zero loads. With calibration all sensors are set to the same reference at a zero load, allowing for consistency in percentage changes once pressure is applied. FIG. 21B displays the three sensors after calibration functioning nearly identical to each other given the same tests when tested separately.

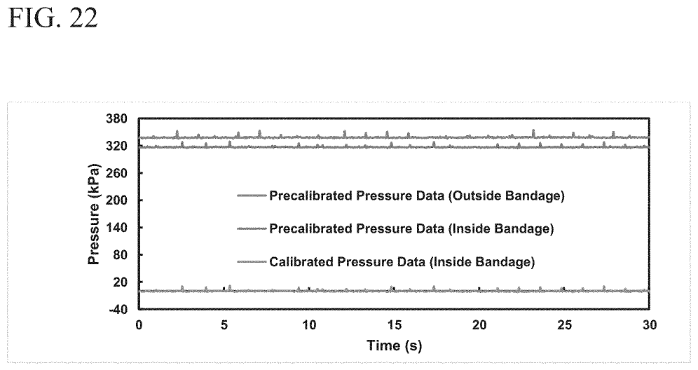

FIG. 22 displays precalibration pressure data of analog-to-digital converter values during calibration based of the smart bandage 100. Since output voltage displayed a change in pressure by wrapping the smart bandage 100, calibration of the sensor 101 is done after the sensor is integrated within the smart bandage. This ensures all recorded changes in pressure will only be detected after the smart bandage 100 is applied to the wound area.

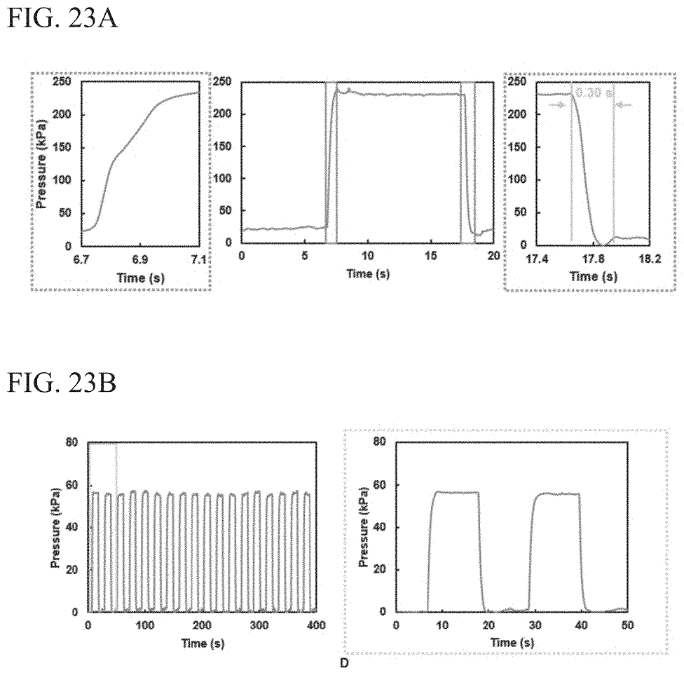

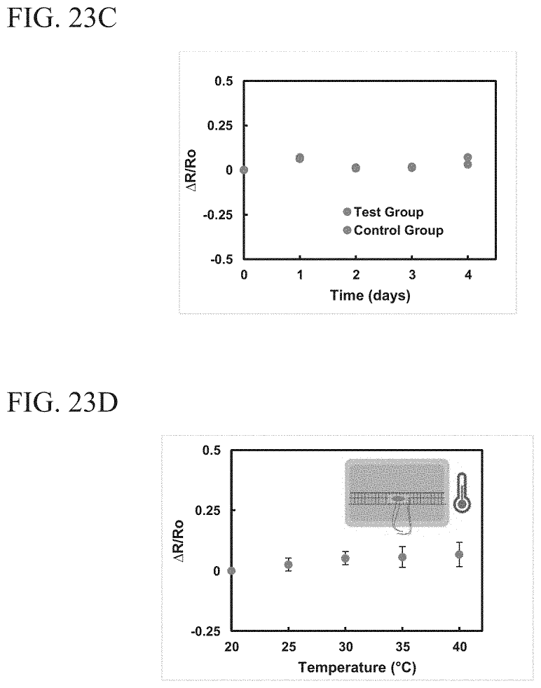

As shown in FIG. 23A-D, a study was performed to determine characterization of the pressure sensor. As shown in FIG. 23A, response time of the pressure sensor 101 of the smart bandage 100 was visualized with extracted views of the onloading and offloading responses. As shown in FIG. 23B, changes in pressure during a period of cyclic loads (n=100) with extracted views of two cycles visualized. As shown in FIG. 23C, fractional changes in resistance of the pressure sensor 101 of the smart bandage 100 over an extended period of complete submersion in water (n=3) are visualized. As shown in FIG. 23D, fractional changes in resistance of the pressure sensor 101 of the smart bandage 100 over a period of increasing temperatures in an oven (n=3) are visualized.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

1. A smart bandage comprising:
a dressing; and
a pressure sensor coupled to the dressing, the pressure sensor configured to be positioned on a wound area while the dressing bandages the wound area and further configured to measure at least one pressure reading at the wound area, the pressure sensor comprising
a force sensitive film configured to generate a pressure signal based on pressure applied thereto, the force sensitive film operably coupled to the dressing such that pressure applied to the wound area is coupled to the force sensitive film via the dressing,
a near field communication device operably coupled to the force sensitive film for reporting the pressure signal; and
a battery configured to provide power to the pressure sensor.

2. The smart bandage of claim 1, wherein the dressing is a foam bandage for a diabetic ulcer.

3. The smart bandage of claim 1, wherein the pressure sensor has thickness of from about 0.1 to about 1 mm, from about 0.2 to about 0.8 mm, or from about 0.3 to about 0.5 mm and the force sensitive film has a thickness of about 0.3 mm or less, about 0.2 mm or less, or about 0.1 mm or less.

4. The smart bandage of claim 4, wherein the force sensitive film has a volume resistivity of about 500 ohm-cm or less and a surface resistivity of about 31,000 ohms/sq·cm or less.

5. The smart bandage of claim 1, wherein the force sensitive film has a diameter about 10 mm to about 40 mm.

6. The smart bandage of claim 1, wherein the force sensitive film comprises an electrically conductive polymer of electrically conductive carbon or carbon black.

7. The smart bandage of claim 1, wherein the force sensitive film comprises a planar metal film with a thickness of about 400 nm or less, about 300 nm or less, or about 200 nm or less.

8. The smart bandage of claim 1, wherein the force sensitive film further comprises a polymer layer.

9. The smart bandage of claim 1, wherein the force sensitive film comprises a plurality of planar metal films and a plurality of polymer layers.

10. The smart bandage of claim 1, further comprising one or more covering layers covering a surface of the force sensitive film of a thickness of about 5 μm to about 250 μm.

11. The smart bandage of claim 10, wherein the covering layers comprise polyimide, SU-8 or polydimethylsiloxane.

12. The smart bandage of claim 10, wherein the force sensitive film is positioned between two or more covering layers.

13. The smart bandage of claim 1, wherein the pressure sensor comprises one or more conductive threads between the force sensitive film and a top covering layer for communication between the pressure sensor and the communication device.

14. The smart bandage of claim 13, wherein a first of the one or more conductive threads is between a top side of the force sensitive film and the top covering layer, and a second of the one or more conductive threads is below a bottom side of the force sensitive film and a bottom covering layer.

15. The smart bandage of claim 1, wherein the pressure sensor detects pressure by a change in a geometry of the force sensitive film which changes a resistance in the force sensitive film.

16. The smart bandage of claim 1, wherein the smart bandage further comprises a porous adhesion layer and the pressure sensor is between the porous adhesion layer and the dressing.

17. The smart bandage of claim 1, wherein the smart bandage is configured to assign the at least one pressure reading to a category selected from the group consisting off 1) adequate offloading, 2) marginal offloading, and 3) inadequate offloading.

18. The smart bandage of claim 17, wherein the smart bandage is configured to send an alert to a user interface if the at least one pressure reading is assigned to inadequate offloading.

19. The smart bandage of claim 1, wherein the smart bandage is configured to continuously measure pressure readings.

20. The smart bandage of claim 19, wherein the user interface maintains a log of the pressure readings.

21. A method of treating a wound area of a patient, the method comprising:
applying the smart bandage of claim 1 to the wound area of the patient;
measuring one or more pressure readings at the wound area using the pressure sensor; and
transmitting the one or more pressure readings to a user interface using the near field communication device.

22. The method of claim 21, wherein applying the smart bandage to a wound area that experiences loading such that the method further comprises reporting an offloading suggestion based on the one or more pressure readings to an end user using the user interface.

23. The method of claim 21, further comprising cutting the smart bandage to a shape corresponding to the wound area or based on the shape of the wound area.

24. The smart bandage comprising:
a dressing;
a pressure sensor coupled to the dressing, the pressure sensor configured to be positioned on a wound area while the dressing bandages the wound area and further configured to measure at least one pressure reading at the wound area, the pressure sensor comprising:
a force sensitive film configured to generate a pressure signal based on pressure applied thereto, the force sensitive film operably coupled to the dressing such that pressure applied to the wound area is coupled to the force sensitive film via the dressing, and
a near field communication device operably coupled to the force sensitive film for reporting the pressure signal;
a user interface configured to display the reported signal to a user for comparing the pressure with a threshold pressure, wherein the threshold pressure is indicative of too much pressure loaded on the wound area; and
a circuit board coupling a connecting housing, general circuit components, data processing and transmission components, and a battery configured to provide power to the pressure sensor;
wherein the connecting housing electrically couples the pressure sensor to the circuit board;
wherein the general circuit components comprise an operational amplifier to amplify and transmit analog data from the pressure sensor to the data processing and transmission components; and
wherein the data processing and transmission components read and extract data from the pressure signal and transmit the data to the user interface.
